(19) United States
(12) Patent Application Publication
(10) Pub. No.: US 2021/0162229 A1
(43) Pub. Date: Jun. 3, 2021

(54) METHOD AND SYSTEM OF PHYSIOTHERAPEUTIC CORRECTION AND THERAPY OF HUMAN SLEEP

(71) Applicant: OBSCHESTVO S OGRANICHENNOI OTVETSTVENNOSTYU "TSENTR NEIROTEKHNOLOGY SNA I BODRSTVOVANIA" (OOO "TSNSI, Moscow (RU)

(72) Inventors: Ilya Sergeevich BLOKHIN, Moscow (RU); Vladimir Borisovich DOROKHOV, g. Mytischi (RU); Alexey Sergeevich BLOKHIN, Moscow (RU)

(21) Appl. No.: 17/050,268

(22) PCT Filed: Apr. 19, 2019
(86) PCT No.: PCT/RU2019/000263

§ 371 Date: Oct. 23, 2020

(30) Foreign Application Priority Data
Apr. 24, 2018 (RU) 2018115258

(51) Int. Cl. A61N 2/00; A61B 5/0205; A61M 21/02; G16H 20/30; G16H 10/20

(52) U.S. Cl. CPC A61N 2/004; A61B 5/0205; A61B 5/024; G16H 20/30; G16H 10/20; A61M 21/02

(57) ABSTRACT

The present invention relates to a method of physiotherapeutic correction and sleep therapy and to a system for its implementation. The invention expands the arsenal of technical means by creating a simple and easy-to-use system which permits an increase in the spectrum of uses. A method according to the invention is implemented using a system comprising a pulse generator (1) designed so as to be capable of generating pulses with an adjustable repetition frequency in a range of from 1 to 40 Hz; a magnetic field shaper (2) connected to the pulse generator; a feedback means (4) intended for tuning the repetition frequency of the generated pulses. Said feedback means can be designed on the basis of a smartphone having an application intended for setting the required correction mode for the repetition frequency of generated pulses before a person goes to sleep or for correcting the repetition frequency of generated pulses on the basis of data from a pulsometer, accelerometer or respirometer which can be arranged on the person or near him/her.

### FIELD OF THE INVENTION

This invention relates to a method of physiotherapeutic correction and therapy of sleep and to a system for its implementation.

### BACKGROUND OF THE INVENTION

There are various devices for normalizing human sleep.

Thus, Russian patent No. 2170109 (publ. Jul. 10, 2001) describes a device for elimination of sleep disorders, wherein a pulsed low-frequency quasistationary electrostatic field is used. This device requires the placement of several electrodes on the human body, which is very inconvenient, since the sleeper often turns.

U.S. Pat. No. 8,517,909 (publ. on Aug. 27, 2013) describes devices and methods for improving sleep, reducing pain and encouraging naturaltreatment. In this solution, a plurality of simultaneous ultra-low-frequency mechanical vibration waves is formed for a patient lying on a table or sitting in an arm-chair. This method can only be used in a hospital or clinic.

Russian patent application No. 94003463 (publ. on Jul. 27, 1996) describes a device for physiotherapeutic influence, intended for rendering the physiotherapeutic assistance in case of pain effects (i.e. for alleviation of pain). The impact is carried out by a rotating permanent magnet, which does not enable it to be used for sleep therapy.

U.S. Pat. No. 5,441,528 (publ. on Aug. 15, 1995) discloses a method and system for applying low-energy emission therapy by means of a high-frequency electromagnetic field with a frequency of 1 GHz, modulated by a low frequency in the range of 0.1 Hz-10 kHz.

The closest analogue of this invention is U.S. Pat. No. 8,808,159 (publ. on Aug. 19, 2014). It describes a device for stimulating local and higher-level autoregulatory mechanisms in the body, it effects by magnetic field pulses with a frequency of 8-15 Hz and an intensity of 30-150 μT. Such magnetic field intensities exceed the hygienic safety standards of the Russian Federation. In addition, this document does not comprise a detailed description of the patented device itself.

### SUMMARY OF THE INVENTION

This invention solves the problem of overcoming the disadvantages of the closest analogue and ensures the achievement of a technical result in the form of an expanding the range of technical means.

In the first object of this invention, a method of physiotherapeutic correction and therapy of human sleep is claimed, comprising generating pulses with a configurable repetition frequency in the range from 1 to 40 Hz; delivering of the generated pulses to the magnetic field former placed at a distance of 50 to 200 cm relative to the said person; adjusting frequency of the pulses based on the feedback from the person.

A feature of the method according to the first object of this invention is that the pulses can be generated of a rectangular shape.

Another feature of the method according to the first object of this invention is that a magnetic field can be created with an intensity of up to 20 μT.

Another feature of the method according to the first object of this invention consists in the fact that the magnetic field can be formed by using three inductor coils placed in three orthogonal planes, one of which is parallel to the plane of the person's bed.

Another feature of the method according to the first object of this invention is that the feedback can be implemented through a mobile application through a survey.

Finally, another feature of the method according to the first object of this invention consists in the fact that the feedback can be performed through a mobile application using at least one of the following means: a heart rate monitor, an accelerometer, a respiration rate meter, wherein the means is placed on or near the person.

In the second object of this invention, a system for physiotherapeutic correction and therapy of human sleep is claimed, comprising: a pulse generator adapted to generate pulses with a configurable repetition frequency in the range from 1 to 40 Hz; a magnetic field former connected to the pulse generator; a feedback means designed to adjust the repetition frequency of the pulses generated.

A feature of the system according to the second object of this invention is that the pulse generator can be configured to generate rectangular pulses.

Another feature of the system according to the second object of this invention is that the magnetic field former can be configured to create a magnetic field with an intensity of up to 20 μT.

Another feature of the system according to the second object of this invention is that the magnetic field former can be based on three inductor coils placed in three orthogonal planes, one of which is parallel to the person's bed plane.

Another feature of the system according to the second object of this invention is that the feedback means can be performed based on a smartphone with a software application designed to set the desired repetition frequency correction mode of the generated pulses during the forthcoming person's sleep.

Finally, another feature of the system according to the second object of this invention consists in the fact that the said feedback means can be implemented based on a smartphone with a software application for correcting the repetition frequency of the generated pulses based on data from at least one of the following means: a heart rate monitor, an accelerometer, a respiration rate meter, wherein the means is placed on or near the person.

### BRIEF DESCRIPTION OF THE DRAWINGS

This invention is illustrated in the figures attached.

FIG. 1 shows a scheme of the system according to the invention.

FIG. 2 shows the signals generated by the system according to the FIG. 1.

### DETAILED DESCRIPTION OF EMBODIMENTS OF THE INVENTION

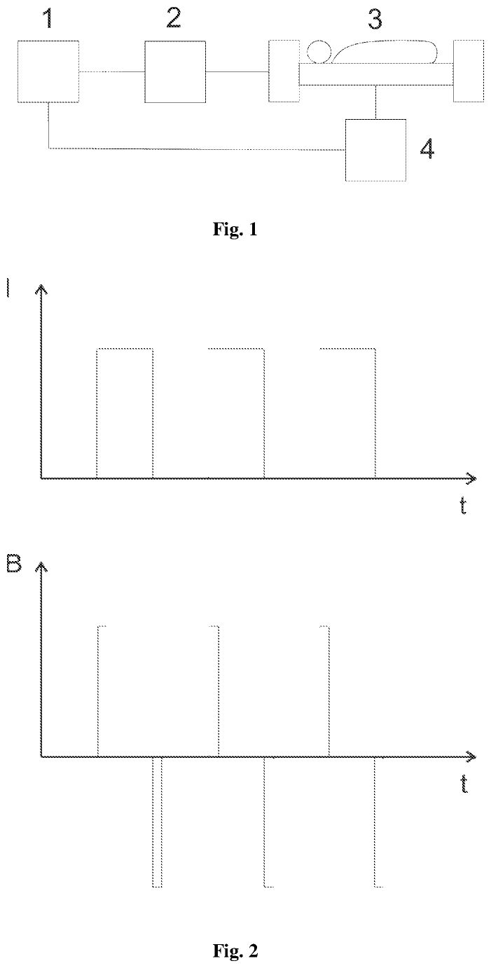

The method according to the first object of the invention can be implemented using the system according to the second object of this invention, the scheme of which is presented in FIG. 1.

The system of physiotherapeutic correction and therapy of sleep comprises a pulse generator 1 configured to generate pulses with a configurable repetition frequency in the range from 1 to 40 Hz, and a magnetic field generator 2 connected to the pulse generator 1. Any suitable device can be used as a pulse generator 1, which is known to those skilled in the art. Preferably, the pulse generator 1 generates rectangular pulses (FIG. 2).

The magnetic field former 2 can be made in the form of an inductor coil placed in a plane, which is parallel to the plane of the bed 3 of the person who needs in a physiotherapeutic correction or therapy of sleep. However, the magnetic field former 3 is preferably made on the basis of three inductor coils placed in three orthogonal planes, one of which is parallel to the plane of the person's bed 3. For example, these three inductor coils can be placed on three adjacent sides of a cube.

The former 3 of the magnetic field is configured to create a magnetic field with an intensity of up to 20 μT, which does not exceed the permissible hygienic safety standards of the Russian Federation. Wherein, in the zone of the bed 3, where a person is located, the intensity of the magnetic field does not exceed 30 nT.

In addition to these components, the system of the invention includes the feedback means 4 for adjusting the pulse repetition frequency from the pulse generator 1. The implementation of the feedback means 4 may be different.

For example, this feedback means 4 can be based on a smartphone with a software application designed to set the required repetition frequency correction mode of the generated pulses during the forthcoming sleep of the person. In this case, feedback can be carried out through a survey. The corresponding software application can be installed on a smartphone or tablet on iOS and Android platforms. The survey will take place in the format of the modified standard questionnaires for the quality of sleep and dreams. That is, after waking up, the user will be asked to answer several control questions. By answering these questions, the application will “understand” the extent of effectiveness of the impact of the system of this invention. And with the accumulation of statistics, this software application will “know” the need of this particular user.

In more advanced versions of the device, a more “rigid” sleep quality control tool will be possible to use. This can be achieved by implementing the feedback means 4 based on a smartphone with a software application for correcting the repetition frequency of the generated pulses based on data from at least one of the following means: a heart rate monitor, an accelerometer, a respiration rate meter. This means can be placed on a person (for example, on his hand) or near him.

The operation of the system according to the second object of this invention for implementing the method according to the first object of this invention is obvious. A magnetic field former 2 is installed near the bed 3 of a person who needs correction or therapy of sleep. The pulse generator 1 generates pulses with a configurable repetition frequency in the range from 1 to 40 Hz. These pulses are fed to the magnetic field former 2 placed at a distance of 50 to 200 cm from the person. The frequency of the pulses generated is adjusted based on the feedback from the feedback means 4.

This invention is based on non-contact remote exposure to weak electromagnetic fields of ultra-low frequency (EMF ULF) which penetrate freely the body tissues. These fields hold a special place among natural environmental factors, which is associated with the interaction of the Earth's atmosphere and the Sun. Unlike other, more high-frequency electromagnetic fields, a noteworthy feature of the EMF ULF range is its coincidence with the frequency characteristics of the bioelectric potentials of organs and tissues of humans and animals, which provides the possibility of “resonant” interaction of EMF ULF with living organisms.

The feedback is preferably implemented using the so-called sleep tracker, which is, for example, a pulse meter+motion sensor. In other cases, a noise analyzer, an eye movement sensor, a brain electroencephalogram recorder can be used. Various sleep trackers are heavily marketed and their specific implementation is not included in the scope of claims of this invention.

Data from the sleep tracker is fed to a mobile application that controls the operation modes of the pulse generator 1 in accordance with the measured parameters of the sleeper. This mobile application indicates the dynamics of the phases of sleep, the dynamics of exposure, as well as local and global statistics. It can accommodate the functions of a “smart alarm clock”, a target scheduler (for example, for developing sleep modes). The mobile application can synchronize data on sleep quality with a remote server, where the user can access the WEB interface and the personal sleep manager, which is the user's personal account in the WEB interface system, which presents his sleep quality data. Based on statistics and goals, the personal manager can create personalized exposure modes for the pulse generator 1 (for example, if the user wants to sleep less and deeper, or, conversely, to see many bright dreams and wake up easily in the morning; if the user wants to change the sleep mode, time of falling asleep and awakening, etc.). A separate function of a personal manager is communication with a personal expert (in accordance with the subscription fees on the WEB interface), who will have access to personal user statistics and solve more complex personal tasks for normalizing sleep.

It should be noted separately that due to the small size of the pulse generator 1 and the magnetic field former 2, the system of this invention is transportable and can be used with an independent power source (for example, in an airplane).

Thus, this invention expands the range of technical means by creating a simple and easy-to-use system that enables to increase the range of uses.

1. A method of physiotherapeutic correction and therapy of human sleep, comprising:
generating pulses with a configurable repetition frequency in the range from 1 to 40 Hz;
delivering of the generated pulses to the magnetic field former, placed at a distance of 50 to 200 cm relative to the person;
adjusting frequency of the said pulses based on feedback with the said person.

2. The method according to claim 1, wherein the said pulses are generated having rectangular shape.

3. The method according to claim 1, wherein the said magnetic field is created with an intensity of up to 20 μT.

4. The method according to claim 1, wherein the said magnetic field is formed by means of three inductor coils placed in three orthogonal planes, one of which is parallel to the plane of the person's bed.

5. The method according to claim 1, wherein the said feedback is implemented through a mobile application through a survey.

6. The method according to claim 1, wherein the said feedback is implemented through a mobile application using at least one of the following means: a heart rate monitor, an accelerometer, a respiration rate meter, wherein the means is placed on or near the said person.

7. A system of physiotherapeutic correction and therapy for human sleep, comprising:
a pulse generator adapted to generate pulses with a configurable repetition frequency in the range from 1 to 40 Hz;
a magnetic field former connected to the said pulse generator;
a feedback means designed to adjust the said repetition frequency of the generated pulses.

8. The system according to claim 7, wherein the said pulse generator is configured to generate rectangular pulses.

9. The system according to claim 7, wherein the said magnetic field former is configured to create a magnetic field with an intensity of up to 20 μT.

10. The system according to claim 7, wherein the said former of the magnetic field is implemented on the basis of three inductor coils placed in three orthogonal planes, one of which is parallel to the plane of the person's bed.

11. The system according to claim 7, wherein the said feedback means are based on a smartphone with a software application designed to set the required repetition frequency correction mode of the generated pulses during the forthcoming sleep of the said person.

12. The system according to claim 7, wherein the said feedback means is based on a smartphone with a software application for correcting the repetition frequency of the generated pulses based on data from at least one of the following means: a heart rate monitor, an accelerometer, a respiration rate meter, wherein the means is placed on or near the said person.

13. The method according to claim 3, wherein the said magnetic field is formed by means of three inductor coils placed in three orthogonal planes, one of which is parallel to the plane of the person's bed.

14. The system according to claim 9, wherein the said former of the magnetic field is implemented on the basis of three inductor coils placed in three orthogonal planes, one of which is parallel to the plane of the person's bed.
